(19) United States
(12) Patent Application Publication
(10) Pub. No.: US 2013/0231629 A1
(43) Pub. Date: Sep. 5, 2013

(54) ATTACHMENT MEMBER WITH COORDINATED GRAPHICS FOR DISPOSABLE ABSORBENT ARTICLES

(71) Applicants: Julie A. Paveletzke, Appleton, WI (US); Katie A. Boland, Neenah, WI (US); Kimberly M. Downs, Oshkosh, WI (US); Kimberly L. Ellefson, Oshkosh, WI (US); Suzanne M. Schmoker, Oshkosh, WI (US)

(72) Inventors: Julie A. Paveletzke, Appleton, WI (US); Katie A. Boland, Neenah, WI (US); Kimberly M. Downs, Oshkosh, WI (US); Kimberly L. Ellefson, Oshkosh, WI (US); Suzanne M. Schmoker, Oshkosh, WI (US)

(21) Appl. No.: 13/412,284

(22) Filed: Mar. 5, 2012

(51) Int. Cl. A61F 13/62

(52) U.S. Cl. 604/386

(57) ABSTRACT

An attachment member for use with absorbent articles is disclosed. The attachment member for a disposable absorbent article has a first side section securely attached to the disposable absorbent article, the first side having a first graphic. The attachment member has a second side releasably attached to the disposable article, the second side having a second graphic. In one embodiment, the first graphic has a first Pattern Complexity Value and the second graphic having a second Pattern Complexity Value. In another embodiment, the first graphic has a first color and the second graphic has a second color, and the first color and the second color are different. The change in appearance of the graphic on the attachment member allows for a user to more easily detect the presence of the releasable edge and allows for adjustment of the fit of the absorbent article.

### BACKGROUND

Absorbent articles for absorbing human discharges can appear similar in size and shape to regular cloth underwear which is designed to be laundered and reused two or more times. A disposable absorbent article is intended to be worn by persons, including infants, toddlers, or adults, and is designed for a single or temporary use and is meant to be disposed of after being used once instead of being laundered or dry cleaned for re-use. Some examples of disposable absorbent articles include infant diapers, training pants, adult incontinence garments, feminine pants, etc.

Some absorbent articles manufactured today resemble regular cloth underwear in that they have a waist opening and a pair of leg openings. Such pant-like disposable absorbent articles can be pulled up around the torso of a wearer in a similar fashion as regular cloth underwear. Still other pant-like disposable absorbent articles have an open or flat configuration and are designed to be placed adjacent to a wearer's torso and then rely upon one or more attachment tabs or fasteners to secure the article around the wearer's torso. This design is beneficial for bed bound users who may be immobile or for children who need assistance in securing the article in place. Still other adjustable, pant-like articles contain attachment means for opening and closing the waist opening after the article has been positioned around the wearer's torso. The adjustable article has an advantage in that the wearer does not have to remove outer clothing in order to check the status of the article or to remove the undergarment from their body.

With certain refastenable articles, such as adult incontinence underwear and enuresis pants, it is important that the garments look and feel as much as possible like “regular” underwear to promote an improved sense of normalcy to the wearer who suffers from incontinence or enuresis. Accordingly, there is a desire in certain cases to incorporate fastening systems in garments in such a way that the fastening system is relatively discreet in order to make the garment more like “regular” cloth underwear. One possible approach is to provide at least one garment-like, refastenable seam, provided by one or more strips of mechanical fastening material, such as a hook-and-loop style fastener. However, one potential drawback to such designs is that a garment-like refastenable seam may be difficult to detect and difficult to manipulate by the user of the product. In particular embodiments, when a fastener which lies relatively close to an edge of the base substrate to which it is applied, it can in some cases be difficult to locate the edge of the refastenable seam, and, once located, it can in some cases be difficult to grasp the edge of the refastenable seam so as to disengage the seam to allow the product to be opened and inspected or removed.

As a result, a disposable absorbent article including a refastenable seam or fastening portion that is garment-like yet easy to see and/or easy to grasp is needed.

### SUMMARY

Generally, an attachment member for use with absorbent articles is disclosed. The attachment member for a disposable absorbent article has a first side section securely attached to the disposable absorbent article, the first side having a first graphic. The attachment member has a second side releasably attached to the disposable article, the second side having a second graphic. In one embodiment, the first graphic has a first Pattern Complexity Value and the second graphic having a second Pattern Complexity Value, wherein the first Pattern Complexity Value and the second Pattern Complexity Value are different. In another embodiment, the first graphic has a first color and the second graphic has a second color, and the first color and the second color are different. In other embodiments, both the Pattern Complexity Value and the color of the first and second graphics are different. The change in appearance of the graphic on the attachment member allows for a user to more easily detect the presence of the releasable edge and allows for adjustment of the fit of the absorbent article.

An absorbent article including an attachment member as described above is also disclosed. Desirably, the first graphic corresponds with the graphic on the disposable absorbent article.

### BRIEF DESCRIPTION

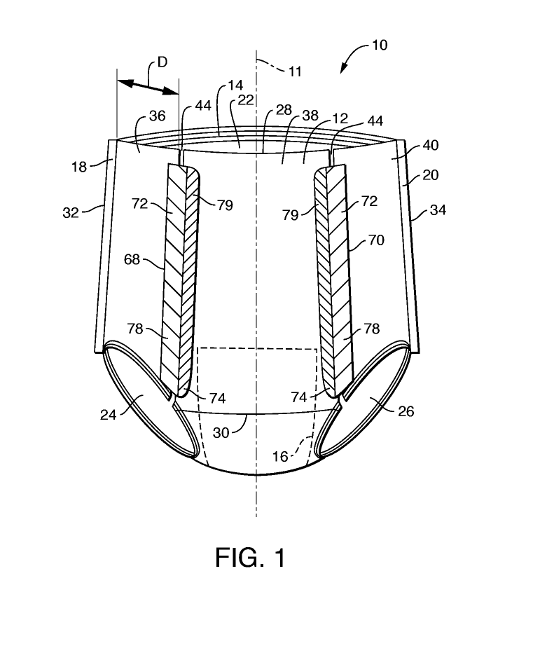

FIG. 1 depicts a front perspective view of one example of an absorbent article, the article shown in a pre-fastened, pant-like configuration.

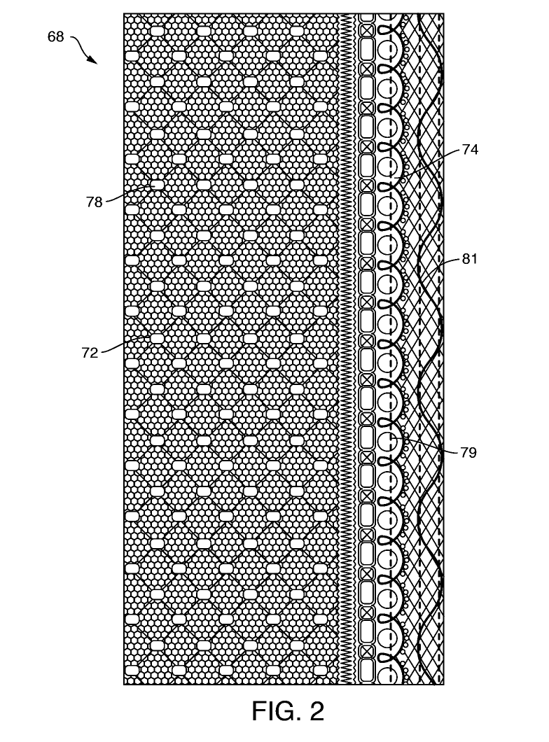

FIG. 2 illustrates one embodiment of the attachment member for use with the absorbent article as described in FIG. 1.

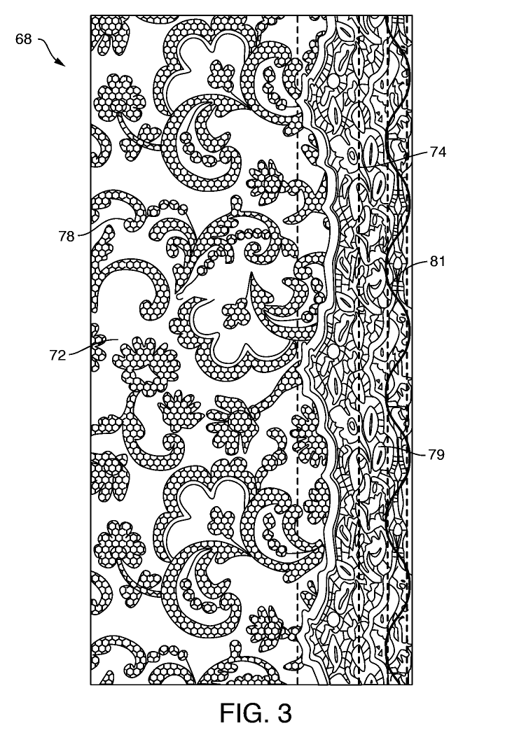

FIG. 3 illustrates another embodiment of the attachment member for use with the absorbent article as described in FIG. 1.

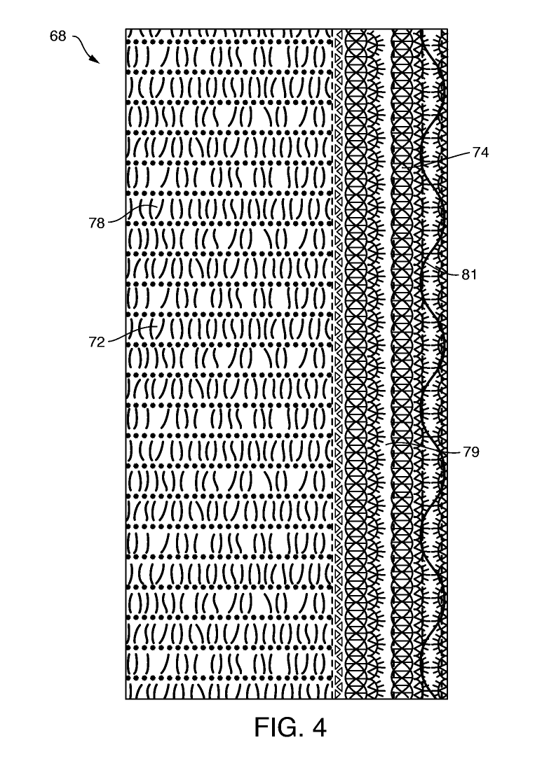

FIG. 4 illustrates another embodiment of the attachment member for use with the absorbent article as described in FIG. 1.

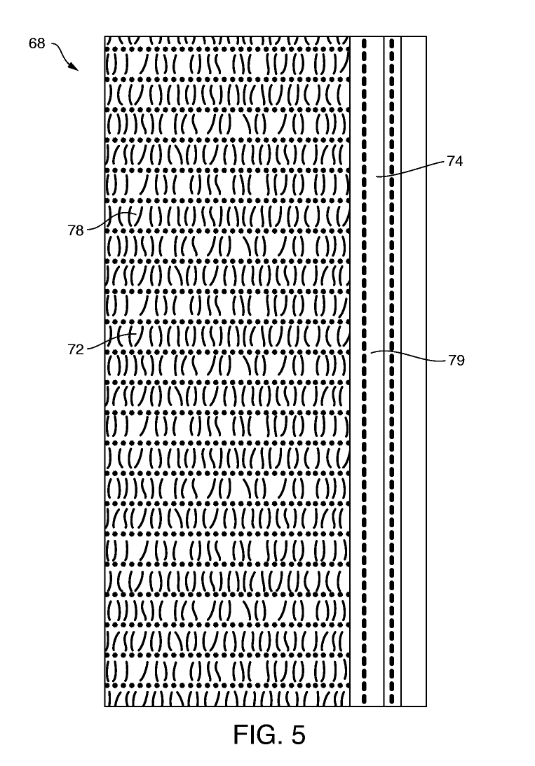

FIG. 5 illustrates another embodiment of the attachment member for use with the absorbent article as described in FIG. 1.

### DETAILED DESCRIPTION

Generally, an attachment member for use with absorbent articles is disclosed. The attachment member for a disposable absorbent article has a first side section securely attached to the disposable absorbent article, the first side having a first graphic. The attachment member has a second side releasably attached to the disposable article, the second side having a second graphic. In one embodiment, the first graphic has a first Pattern Complexity Value and the second graphic having a second Pattern Complexity Value, wherein the first Pattern Complexity Value and the second Pattern Complexity Value are different. In another embodiment, the first graphic has a first color and the second graphic has a second color, and the first color and the second color are different. In other embodiments, both the Pattern Complexity Value and the color of the first and second graphics are different. The change in appearance of the graphic on the attachment member allows for a user to more easily detect the presence of the releasable edge and allows for adjustment of the fit of the absorbent article. Reference to the Figures shall be made in describing various embodiments. It should be noted that the embodiments depicted in the Figures and described herein are merely representative examples. The various embodiments are suitable for use in conjunction with disposable absorbent articles such as refastenable adult incontinence underwear, pre-fastened disposable diapers, refastenable disposable training pants or swim pants, refastenable disposable enuresis garments, and the like. For illustration purposes, various embodiments shall be described in conjunction with refastenable incontinence or enuresis underwear.

Within the context of this specification, each term or phrase below will include the following meaning or meanings.

“Attached” refers to the joining, adhering, bonding, connecting, or the like, of two elements. Two elements will be considered to be attached together when they are attached directly to one another or indirectly to one another, such as when each is directly attached to intermediate elements.

“Disposable” refers to articles which are designed to be discarded after a limited use rather than being laundered or otherwise restored for reuse.

“Elastomeric” refers to a material or composite which can be elongated by at least 50% of its relaxed length and which will recover, upon release of the applied force, at least 20% of its elongation. It is generally preferred that the elastomeric material or composite be capable of being elongated by at least 100%, more preferably by at least 200%, of its relaxed length and recover, upon release of an applied force, at least 50% of its elongation.

“Longitudinal” and “transverse” have their customary meaning, as indicated by the longitudinal and transverse axes depicted in the Figures. The longitudinal axis lies in the plane of the article and is generally parallel to a vertical plane that bisects a standing wearer into left and right body halves when the article is worn. The transverse axis lies in the plane of the article generally perpendicular to the longitudinal axis.

These terms may be defined with additional language in the remaining portions of the specification.

Referring to FIG. 1, an adjustable pant-like disposable absorbent article 10 having a longitudinal axis 11 is shown. The adjustable pant-like disposable absorbent article 10 is designed to absorb liquid, semi-solid and/or solid waste discharged from a human being. A “disposable absorbent article” as used herein is an article that is intended to be worn by persons, including infants, toddlers or adults, which is designed for a single or temporary use and is meant to be disposed of after being used once instead of being laundered or dry cleaned for re-use. The adjustable pant-like disposable absorbent article 10 is designed to absorb and/or retain one or more bodily discharges of waste material such as urine, perspiration, excrement, feces, menses, menstrual fluid, as well as other liquid and/or solid waste.

The adjustable pant-like disposable absorbent article 10 includes a front waist region 12, a back waist region 14 and a chassis including absorbent assembly 16 secured to the front and back waist regions, 12 and 14 respectively. The front and back waist regions, 12 and 14 respectively, are joined together by a pair of seams 18 and 20 to form a waist opening 22 and a pair of leg openings 24 and 26.

The front waist region 12 and/or the back waist region 14 can be formed from a single piece of material or they can be formed as a laminate consisting of two or more layers. The layers of the laminate can be of the same material or different material. In one embodiment, a laminate is formed from a first layer and a second layer. Sandwiched between the first and second layers are two or more elastic strands. Desirably, from two to about a hundred elastic strands can be utilized in either the front or back waist regions, 12 and 14, depending upon the overall size of each panel. The elastic strands 56 can be formed from LYCRA, or a similar material. The diameter and/or cross-sectional configuration of the elastic strands 56, the decitex (weight in grams per 10,000 meters) of the elastic strands, and the tension imparted into the elastic strands 56 can all be varied to suit one's particular product needs. The number of elastic strands present in the front waist region 12 can be less than, equal to or greater than the number of elastic strands present in the back waist region 14. The exact number of elastic strands that are utilized should be sufficient to ensure that the disposable absorbent article 10 snuggly conforms to the wearer's torso.

The elastic strands can be coated with an adhesive. By adhesively coating each of the elastic strands, instead of slot coating a major portion of the inner surface of at least one of the first and second layers, softer front and back waist region, 12 and 14 respectively, can be obtained. Wearers of disposable absorbent articles prefer a product that has a softer feel since it is more underwear like.

In other embodiments, the elastomeric nonwoven material comprises an elastomeric film sandwiched between two nonwoven facing layers. U.S. Pat. No. 7,803,244 to Siqueira et al., hereby incorporated by reference, discloses particular examples of elastomeric nonwoven composites suitable for use in the adjustable article described herein.

It should be noted that the front and/or back waist regions, 12 and/or 14 respectively, can be formed from a breathable or a non-breathable material. Desirably, the front and back waist region, 12 and 14 respectively, are formed from a breathable material or a material that is treated or processed to be breathable. Spunbond and bonded carded webs are two breathable materials that work well as front and back waist regions, 12 and 14 respectively, in disposable absorbent articles. Bonded carded webs are produced and commercially sold by a variety of vendors. Other materials that can be used to form the front and back waist regions, 12 and 14 respectively, include woven and non-woven materials formed from natural or synthetic fibers; polyolefins, such as polypropylene or polyethylene; thermoplastic films; as well as other materials known to those skilled in the art. A metallocene polypropylene works very well since it has a soft feel and can be easily ultrasonically bonded to itself.

In the embodiment shown, the front waist region 12 includes a waist edge 28, a crotch edge 30 and a pair of side edges 32 and 34. In a three piece construction wherein an absorbent assembly 16 is secured between the front waist region 12 and the back waist region 14, the crotch edge 30 is well defined. In absorbent articles of a different construction, the crotch edge 30 can be an imaginary line transversely drawn between the pair of leg openings 24 and 26 at a location where one considers the front waist region 12 to end. The exact size and configuration of the front waist region 12 can vary to suit one's particular needs. The front waist region 12 has a first side section 36, a middle region 38 and a second side section 40. The front waist region 12 is completely severed between both the first side section 36 and the middle section 38 and the second side section 40 and the middle section 38 creating a first fully severed region, or gap, 42 and a second fully severed region 44. The first side section 36 is aligned adjacent to the side edge 32 at its leading edge forming the seam 18 and the second side section 40 is aligned adjacent to the side edge 34 at its leading edge forming the seam 18. The middle section 38 has a first edge and a second edge and is located between the first and second sections, 36 and 40 respectively. The terminal edge of the first side section is aligned adjacent to the first edge of the middle section 38 and the terminal edge of the second side section 40 is aligned adjacent to the second edge of the middle section 38. As depicted, the middle section 38 is centrally located and is bifurcated by the longitudinal axis 11 at a midpoint of the front waist region 12. In alternative embodiments, the fully severed regions, 42 and 44, may be perforated lines to allow for a user to break the perforations and create an adjustable severed region.

The first and second fully severed regions, 42 and 44, can be linear or non-linear in configuration. In FIG. 1, the first and second fully severed region, 42 and 44, are shown having a linear or straight configuration. The first and second fully severed regions, 42 and 44, extend longitudinally from approximately the waist edge 28 down to approximately one of the leg openings 24 or 26. The first and second fully severed regions, 42 and 44, can be aligned parallel to the longitudinal axis 11 or be angled thereto. For example, the first and second fully severed cuts, 42 and 44, can be tapered relative to the longitudinal axis 11, if desired. In addition, the fully severed cuts, 42 and 44, may also be curved.

Still referring to FIG. 1, each of the first and second fully severed regions, 42 and 44, extend from approximately the waist edge 28 of the front waist region 12 to one of the pair of leg openings 24 and 26. Another way of describing this is to say that the first and second fully severed regions, 42 and 44, extend from approximately the waist edge 28 of the front waist region 12 to approximately the crotch edge 30 of the front waist region 12.

Still referring to FIG. 1, each of the first and second fully severed regions, 42 and 44, is shown being aligned parallel to one of the pair of seams 18 and 20. Such an arrangement provides for an aesthetically pleasing appearance to the front waist region 12.

Desirably, in the pre-fastened condition, the first side sections 36 and the middle section 38 of the front waist region 12 do not overlap, and the second side section 40 and the middle section 38 of the front waist region 12 do not overlap in the severed regions 42, 44. In one exemplary embodiment, a first gap may be formed in the first severed region 42 between the first side section 36 and the middle section 38 of the front waist region 12 in the pre-fastened condition. In this embodiment, a second gap may be formed in the first severed region 44 between the second side section 40 and the middle section 38 of the front waist region 12 in the pre-fastened condition. In another exemplary embodiment, a terminal or outer edge of the first side section 36 abuts against the first edge of the middle section 38 of the front waist region 12 in the pre-fastened condition. In this embodiment, a terminal or outer edge of the second side section 40 abuts against the second edge of the middle section 38 of the front waist region 12 in the pre-fastened condition.

Referring again to FIG. 1, the front waist region 12 further includes a pair of attachment members 68 and 70. Each attachment member 68 and 70 includes a first side portion 72 and a second side portion 74. The first side portion 72 of the attachment member 68 can be permanently secured to the first region 36 of the front waist region 12 and the first side portion 72 of the other attachment member 70 can be permanently secured to the third region 40 of the front waist region 12. Alternatively, it should be evident to those skilled in the art that the first portion 72 of each of the attachment members 68 and 70 could be securely attached to the middle section 38.

The first side portion 72 of each attachment member has a first graphic 78. The second side portion 74 of each attachment member has a second graphic 79. For purposes herein, a “graphic” means a pattern or color created by printing or colored laminate. When patterns or graphics are printed on the attachment member, the color may be printed on the outer surface, on an inner surface or between surfaces of the attachment member. For example, if the attachment member is a multilayer structure, the color or pattern may be printed between the layers or on the outer layer. When printing attachment members, the color or pattern may be applied to the outside surface of the attachment member, on the inside surface of the attachment member, or between layers of the attachment member if the attachment member has multiple layers.

As used herein, the term “color” is intended to mean an individual's perception of the spectral composition of visible light coming from a portion of an object. Color characteristics include hue, saturation and luminosity. Each is a separate color characteristic. Hue is the attribute of a color which allows it to be classified as a given color. Saturation, which is sometimes referred to as vividness, is the intensity of the color. Saturation is the degree of freedom from gray. Luminosity, sometimes referred to as value, is the degree of lightness (paleness) or darkness in a color. For example, a blue with white added is a pale color, e.g., baby blue, and blue with black added is a dark color, e.g., navy blue.

As used herein, the term “pattern” is used to describe the individual's perception of spatial variation of visible light due to contrasts in spatial variation of light due to the color, form, and texture of a portion of an object incorporated into the object by the manufacture of the elements. This contrast creates various visual distinct regions or lines sometimes referred to as “figures” within its surroundings sometimes referred to as “background.” Patterns can be formed by combinations of contrasting color, form, and texture relative to its surroundings. An element can have more than one pattern, but each pattern would be distinguishable, recognizable, and separate from the other patterns on the element. Pattern is also a term used to describe the observer's perception of combined effects of more than one color, form, or texture within a portion of an observer's field of view.

Desirably, the first graphic 78 on the first side portion 72 of the attachment member 68, 70 is different than the second graphic 79 on the second side portion 74 of the attachment member 68, 70. The different graphic on the second side portion provides a cue to the user of the releasable second side portion 74 of the attachment member so that the user may easily access the attachment member and readjust the fit of the article.

In one embodiment, the first graphic 78 has a first pattern and the second graphic 79 has a different second pattern. In some embodiments, the second graphic is more complex than the first graphic 78, and, in other embodiments, the second graphic 79 is less complex the first graphic 78. Each pattern has a Pattern Complexity Value as described and calculated below in the Test Method section. Desirably, the first graphic has a first Pattern Complexity Value and the second graphic having a second Pattern Complexity Value, wherein the ratio of the second Pattern Complexity Value to the first Pattern Complexity Value is between 1:10 and 50:1. More desirably, the ratio of the second Pattern Complexity Value to the first Pattern Complexity Value is at 3:2 and 20:1.

In another embodiment, the first graphic 78 includes a first color and the second graphic 79 has a different second color. Each pattern has a Color Difference Value as described and calculated below in the Test Method section. Desirably, a Color Difference Value between the first side and second side is at least 2.0. More desirably, a Color Difference Value between the first side and second side is between 3 and 20.

In some embodiments, the same color may be used in both the first side section 72 and the second side section 74 with different patterns in each side section. In other embodiments, the same patterns may be used in both the first side section 72 and the second side section 74 with different colors in each side section 72, 74. In still other embodiments, a different pattern and a different color may be used in each side section 72, 74. In still other embodiments, multiple colors and patterns may be used in each side section 72, 74. However, the first graphic 78 must also be contrasted against the second graphic 79 by color or pattern to provide a distinction and cue to the user. In still other embodiments, the pattern on the first graphic and the second graphic is the same, but the size of the pattern changes.

In some embodiments, the graphics 78, 79 on the attachment members 68, 70 coordinate with graphics on the remainder of the front panel region of the absorbent article. Desirably, the first graphic 78 matches the color and pattern of the absorbent article 10 to allow the attachment member 68, 70 to blend in with the rest of the article and look more underwear like while allowing for the second graphic to contrast and provide a distinction and cue to the user. In another embodiment, the color of the fastener 68, 70, corresponds with the color of the remainder of the absorbent article.

As illustrated in FIGS. 2-5, the second side 74 of the attachment member 68, 70 may include a shaped area 81. It should be understood that the term “shaped area” refers to an edge that is unattached after the disposable article is opened. The terminal edge can be a single layer cut or formed edge, or can include a double-layer folded edge, or can include an edge formed by a plurality of layers. Desirably, the terminal edge is scalloped as illustrated. The shaped area 81 may be formed by cutting into a shaped edge. Alternatively the shaped area may be formed by embossing or printing on the article.

In the embodiment shown in FIG. 1, the first side portions 72 can be “securely attached” using an adhesive, heat, pressure, a combination of heat and pressure, an ultrasonic bond, a chemical bond or by other means known to those skilled in the art.

While not required, each of the second side portions 74 of the attachment members 68 and 70 are depicted as extending almost the entire length of the fully severed portions 42 and 44. This means at least 50% of the distance between the waist end and the leg opening are covered by the attachment member. Desirably, the first attachment member and second attachment member extend at least 75% of the distance between the waist end and the leg opening. More desirably, the first attachment member and second attachment member completely extend at least 95% of the distance between the waist end and the leg opening. The second side portions 74 of the attachment member 68 can bridge across the first fully severed region 42 and the second side portion 74 of the other attachment member 70 can bridge across the second fully severed region 44. By extending substantially the entire length of and bridging the fully severed portions, 42 and 44, the attachment members cover the fully severed portions and provide a more underwear like appearance.

The second side portions 74 of the attachment members 68 and 70 can be releasably attached to the middle section 38 of the front waist region 12. Alternatively, the second side portions 74 of the attachment members 68 and 70 can be releasably attached to another region of the side sections of the front waist region 12. The attachment member 68, 70 may be constructed of a non-extensible or elastomeric material. Referring now to FIG. 1, each of the second side portions 74 of the attachment members 68 and 70 has an inner surface that contains a fastener. The fastener can be a mechanical fastener. Desirably the fastener is a hook fastener. In FIGS. 2 and 3, the fastener 82 is shown as a plurality of fine hooks, such as VELCRO hooks. VELCRO is a trademark of Velcro USA, Inc. (Manchester, N.H.). The hooks are designed to easily engage and be removed from a material wherein the material has a loose weave pattern or the fibers forming the material will allow the hooks to be attached to them. The mating material is commonly referred to as the loop member of a hook and loop fastener. The middle section 38 of the front waist region 12 should be formed of such a material. When the hooks engage into the middle section 38, a secure but releasable fastener is formed. The hooks can be easily removed from the loop material by pulling the edge of the fastener outward away from the middle section 38. Alternatively, as illustrated in FIGS. 2 and 3, a loop material 83 or loose fibers may be placed onto the middle section 38 to facilitate the fastening component. Therefore, hook and loop fasteners are referred to as being releasable and can be fastened and released several times. In alternative embodiments, the mechanical fastener constructed of hooks may be placed on to the middle section 38 of the front body panel 12. In this embodiment, the fastening component constructed of a loop material or loose fibers would be placed on the attachment member 68, 70. Both the mechanical fastener and the fastening component may be integral or separately attached.

FIG. 1 depicts the pair of attachment members 68 and 70 being securely fastened to the middle section 38 of the front waist region 12. If the wearer of the disposable absorbent article 10 or a caregiver wishes to inspect the article 10, the user would open the pair of attachment members 68 and 70 to the position shown in FIG. 2. Since the first and fully severed portions 42 and 44 are already broken, the middle section 38 of the disposable absorbent article 10 can be easily moved outward away from the wearer's torso. The wearer can then inspect the absorbent assembly 16 to see if it needs to be changed. If so, the disposable absorbent article 10 can be removed from about the wearer's torso and be replaced by another article. If the absorbent assembly 16 is still capable of accepting additional body fluid, the middle section 38 is moved back against the wearer's torso and the pair of attachment members 68 and 70 is refastened to the middle section 38.

In an alternative embodiment, the front waist region 12 extends the entire width of the absorbent article 10 and may be attached to the back waist region 14 by the attachment members 68 and 70. In another embodiment, the front waist region 12 extends the entire width of the absorbent article 10, is bonded by side seams 18 and 20 and has attachment members 68 and 70 located near the side seams 18, 20.

### Test Methods

### Complexity Value

The complexity of a given graphic can be determined by using the Pattern Complexity Value (PCV or complexity value) measurement method described herein. Generally, the Pattern Complexity Value (PCV) method determines a numeric value of complexity for a printed graphic pattern via a combination of specific image analysis measurement parameters. The PCV method is performed using conventional optical image analysis techniques to detect graphic patterns and measure the complexity of the graphic patterns when viewed using a camera with incident lighting. An image analysis system controlled by an algorithm detects and measures several of the dimensional properties of the graphic pattern. The resulting dimensional measurement data are combined to calculate the PCV of a given pattern.

The method for determining the PCV of a given sample includes the step of acquiring the image of the sample. An exemplary setup for acquiring the image is representatively illustrated in FIG. 2. Specifically, a CCD video camera 102 (e.g., a Leica DFC 310 FX video camera available from Leica Microsystems of Heerbrugg, Switzerland) is mounted on a standard support 104 such as a Polaroid MP-4 Land Camera standard support available from Polaroid Resource Center in Cambridge, Mo. The standard support 104 is attached to a macro-viewer 106 such as a KREONITE macro-viewer available from Dunning Photo Equipment, Inc., having an office in Bixby, Okla. An auto stage 108 is placed on the upper surface of the macro-viewer 106. The auto stage 108 is used to move and adjust the position, via a joystick, of a given sample 110 for optimal viewing by the camera 102. A suitable auto stage is Model H112, available from Prior Scientific Inc., having an office in Rockland, Mass.

The sample 110 possessing a printed graphic design is placed on the auto stage 108 of a Leica Microsystems QWIN Pro Image Analysis system, under the optical axis of a 20 mm Nikon AF Nikkor lens 112 with an f-stop setting of 4. The Nikon lens 112 is attached to the Leica DFC 310 FX camera 102 using a c-mount adaptor. The distance from the front face of the Nikon lens 112 to the sample 110 is approximately 43 cm. The sample 110 is flattened and any wrinkles removed by covering it with a transparent glass plate and/or fastening it to the auto stage 108 surface using transparent adhesive tape at its outer edges. The sample 110 is illuminated with incident incandescent lighting using four, 150 watt, GE Reflector Flood lamps 114. The lamps 114 are attached to the KREONITE macro-viewer 106. The illumination level of the lamps is controlled with a POWERSTAT Variable Auto-transformer, type 3PN117C, available from Superior Electric, Co. having an office in Bristol, Conn.

The image analysis software platform used to acquire images and perform the dimensional measurements is a QWIN Pro (Version 3.5.1) available from Leica Microsystems, having an office in Heerbrugg, Switzerland. Prior to executing the algorithm below, the method for determining the PCV includes the step of shading correction. Additionally, if the sample includes colored graphics then color white balancing is undertaken and three command lines in the algorithm below (denoted with superscript tt) are changed to reflect color imaging in either red-green-blue (RGB) or hue-saturation-intensity (HSI) color space. Both the shading correction and the white balancing steps are performed using the QWIN software and a flat white background (e.g., a photographic positive from Polaroid 803 film) being illuminated by the flood lamps. The system and images are also accurately calibrated using the QWIN software and a standard ruler with metric markings at least as small as one a millimeter. The calibration is performed in the horizontal dimension of the video camera image.

Thus, the method for determining the PCV of a given sample also includes the step of performing the dimensional measurements. Specifically, an image analysis algorithm is used to acquire and process images as well as perform measurements using Quantimet User Interactive Programming System (QUIPS) language. The image analysis algorithm is reproduced below.

NAME = Pattern Complexity - 1a
PURPOSE = Measures ‘complexity’ of Patterns and Elements via various shape parameters
CONDITIONS = DFC 310 FX; monochrome or color; 20-mm Nikkon (f/4); 4-floods; white or

black back.; cover plate; MP4 pole=69 cm

```
Open File  ( C:\Data\29993\data.xls, channel #1 )
Open File  ( C:\Data\29993\feature data.xls, channel #2 )
REPLICATE = 0
SAMPLE = 0
SET-UP
-- Calvalue = 0.149 mm/px
CALVALUE = 0.149
Calibration  ( Local )
Enter Results Header
File Results Header  ( channel #1 )
File Line  ( channel #1 )
File Line  ( channel #1 )
File Results Header  ( channel #2 )
File Line  ( channel #2 )
File Line  ( channel #2 )
Measure frame  ( x 31, y 61, Width 1330, Height 978 )
Image frame  ( x 0, y 0, Width 1392, Height 1040 )
For  ( SAMPLE = 1 to 1, step 1 )
  PauseText  ( “Enter object classification (e.g. geo, element, pattern, etc.).” )
  Input  ( TITLE$ )
  File  ( TITLE$, channel #1 )
  File Line  ( channel #1 )
  File  ( TITLE$, channel #2 )
  File Line  ( channel #2 )
  File  ( “Object ID”, channel #1 )
  File  ( “Area”, channel #1 )
  File  ( “Perimeter”, channel #1 )
  File  ( “Area Fract.”, channel #1 )
  File Line  ( channel #1 )
  File  ( “Object ID”, channel #2 )
  File  ( “Area”, channel #2 )
  File  ( “Conv. Area”, channel #2 )
  File  ( “Perim.”, channel #2 )
  File  ( “Conv. Perim.”, channel #2 )
  File  ( “Number”, channel #2 )
  File Line  ( channel #2 )
  For  ( REPLICATE = 1 to 5, step 1 )
  Image frame  ( x 0, y 0, Width 1392, Height 1040 )
  Binary Edit  ( Clear Binary2 )
  IMAGE ACQUIRE
  ACQOUTPUT = 0
  Colour Transform  ( Mono Mode ) tt
  PauseText  ( “Position sample for imaging.” )
  Display  ( Image0 (on), frames (on,on), planes (off,off,off,off,off,off), lut 0, x 0, y 0, z 1,
        Reduction off )
  Image Setup DC Twain [PAUSE]  ( Camera 1, AutoExposure Off, Gain 0.00, ExposureTime
        78.43 msec, Brightness 0, Lamp 49.99 ) tt
  Acquire  ( into Image0 )
  ACQFILE$ = “C:\Images\29993 - Hopkins\”+TITLE$+“_”+STR$(REPLICATE)+“.tif”
  Write image  ( from ACQOUTPUT into file ACQFILE$ )
  Display  ( Colour0 (on), frames (on,on), planes (off,off,off,off,off,off), lut 0, x 0, y 0, z 1,
        Reduction off )
  DETECTION AND IMAGE PROCESSING
  PauseText  (“Adjust detection to include all printed areas. If necessary, this line can be
        changed to HSI or RGB detection thru editing.”)
  Detect [PAUSE]  ( blacker than 183, from Image0 into Binary0 delineated ) tt
  PauseText  ( “Is additional/unique image processing required? If yes, enter 1.” )
  Input  ( PROCESS )
  If  ( PROCESS=1 )
    PauseText  ( “Use Binary Amend to optimize detection. The final step must output to
Binary0.” )
    Binary Amend  [PAUSE]  ( Open from Binary0 to Binary0, cycles 1, operator Disc, edge
erode on )
    PauseText  ( “Use Binary Editing to optimize detection. The final step must output to
        Binary0.” )
    Binary Edit [PAUSE]  ( Reject from Binary0 to Binary0, nib Fill, width 2 )
  Else
    Goto CONTINUE
  Endif
  CONTINUE:
  Display  ( Image0 (on), frames (on,on), planes (0,off,off,off,off,off), lut 0, x 0, y 0, z 1,
Reduction off )
  PauseText  ( “Set Measure Frame to encompass features of interest and image frame to be
just inside the measure frame.” )
  Measure frame [PAUSE]  ( x 31, y 61, Width 1330, Height 978 )
  Image frame [PAUSE]  ( x 0, y 0, Width 1392, Height 1040 )
  PauseText  ( “If detected regions are within the image frame only, click on ‘OK.’” )
  Binary Edit  [PAUSE]  ( Cut from Binary0 to Binary1, nib Fill, width 1 )
  Binary Logical  ( C = A AND B : C Binary2, A Binary0, B Binary1 )
  MEASURE FIELD
  MFLDIMAGE = 2
  Measure field  ( plane MFLDIMAGE, into FLDRESULTS(5), statistics into
    FLDSTATS(7,5) ) Selected parameters: Area, Perimeter, Area Fract
  AREA = FLDRESULTS(1)
  PERIM = FLDRESULTS(4)
  AREAFRACT = FLDRESULTS(5)
  File  ( REPLICATE, channel #1, 0 digits after ‘.’ )
  File  ( AREA, channel #1, 1 digit after ‘.’ )
  File  ( PERIM, channel #1, 1 digit after ‘.’ )
  File  ( AREAFRACT, channel #1, 1 digit after ‘.’ )
  File Line  ( channel #1 )
  MEASURE FEATURES
  Feature Expression  ( UserDef1 ( all features ), title Area/Perim =
PAREA(FTR)/PPERIMETER(FTR) )
  Measure feature  ( plane Binary2, 8 ferets, minimum area: 6, grey image: Image0 )
    Selected parameters: Area, X FCP, Y FCP, Perimeter, ConvxPerim,
    ConvexArea
  File  ( REPLICATE, channel #2, 0 digits after ‘.’ )
  FSAREA = Field Sum of ( PAREA(FTR) )
  File  ( FSAREA, channel #2, 1 digit after ‘.’ )
  FSCONVAREA = Field Sum of ( PCONVAREA(FTR) )
  File  ( FSCONVAREA, channel #2, 1 digit after ‘.’ )
  FSPERIM = Field Sum of ( PPERIMETER(FTR) )
  File  ( FSPERIM, channel #2, 1 digit after ‘.’ )
  FSCONVPERIM = Field Sum of ( PCONVPERIM(FTR) )
  File  ( FSCONVPERIM, channel #2, 1 digit after ‘.’ )
  FSNUMBER = Field Sum of ( PACCEPTED(FTR) )
  File  ( FSNUMBER, channel #2, 0 digits after ‘.’ )
  File Line  ( channel #2 )
  Binary Edit  ( Clear Binary2 )
  Next  ( REPLICATE )
  File Line  ( channel #1 )
  File Line  ( channel #2 )
Next  ( SAMPLE )
Close File  ( channel #1 )
Close File  ( channel #2 )
END
```

tt—Denotes command lines that must be changed to their color equivalent format prior to execution if color imaging and detection will be performed.

The QUIPS algorithm is then executed using the QWIN Pro software platform. The analyst is initially prompted to enter in sample identification information. This is followed by a prompting to enter in a base file name for saving the sample replicate specimen images. An opportunity is then given to set up and position the specimen of interest on the sample stage or platform beneath the camera. For most printed designs, a flat white background is suitable to detect the pattern in either gray-scale or color. In some cases, when the specimen is composed of a polymer film, a black background behind the flattened and unwrinkled specimen is suitable to obtain good detection of the pattern.

The sample is positioned so the longest dimension runs horizontally in the image, and the light illumination level of the four-flood lamps is adjusted using the POWERSTAT Variable Auto-transformer to obtain a white level reading of approximately 0.95. During this process of light adjustment, the QUIPS algorithm automatically displays the current white level value within a small window on the video screen. The algorithm then acquires and saves the image to a designated location—typically on the computer's hard drive. The analyst is then prompted to adjust the detection threshold in order to obtain the optimal detection that is possible. The delineation should be turned ‘on’ and the detection interactive window gray-scale histogram as well as visual observation should be used to ensure the best detection possible. For most printed designs, detection will be adjusted in ‘black’ mode in gray-scale or hue-saturation-intensity or red-green-blue mode in color. For polymer film patterns when using a black background, the gray-scale mode will likely need to be switched to ‘white’ mode.

After detection, the analyst is asked whether additional binary image processing is required to further optimize pattern detection. If the analyst believes additional processing will be beneficial, a value of ‘1’ is entered into the prompting window and the analyst is given two opportunities to optimize the binary detection to match the design to the extent possible. In order to check for detected fit versus the actual pattern, the analyst can toggle the ‘control’ and ‘B’ keys on the keyboard simultaneously to turn the overlying binary image on and off. A fit is considered good when the binary image closely matches with the printed pattern with respect to its boundaries and regions within said boundaries. If no additional processing is required, the analyst clicks ‘OK’ without entering any value into the prompting window. If ‘1’ is entered for additional processing, the first opportunity will be thru a ‘Binary Amend’ window showing various options such as ‘closing’ and ‘opening.’ The analyst can experiment to find a good option by changing the output to binary1 or higher. When a specific processing step(s) have been identified, the analyst must do so in such a way so that the final output is into binary0. The second binary ‘edit’ processing step allows for a selection of manual interactions (e.g. reject, accept, draw, etc.) with the image to clean it up for the measurement step of the algorithm. Again, the final step within the manually editing processing step must go into binary0 for the output. If no editing is required, the analyst clicks ‘OK’ and allows the algorithm to proceed.

After the option of additional processing, the algorithm will then prompt the analyst to manually select both measurement and image frame regions of interest (ROI). First, the measurement frame is selected to enclose the detected pattern over as much of the sample (e.g., framing region or focal region) as possible or at least enough to cover one unit cell if there is a pattern that repeats. Secondly, the image frame is selected to be just inside the boundaries of the previously selected measurement frame. The resulting image frame size should be two pixels less wide and long as the measurement frame and located within the measurement frame boundaries.

After the measurement and image frames have been selected, the algorithm will automatically perform measurements and output the data into two different spreadsheets. The first spreadsheet is labeled “data.xls” and is for the field data. The second spreadsheet is labeled “feature data.xls” and is for feature data. The following primary measurement parameter data will be located in the feature data.xls file after measurements and data transfer has occurred:

Area

Perimeter

Convex area

Convex perimeter

Number of features

The following primary measurement parameter data will be located in the data.xls file after measurements and data transfer has occurred. The field area and perimeter data located in the data.xls file are not used for calculations and should be within approximately 5% of those in the featuredata.xls file and may be used to collaborate the accuracy of the comparable area and perimeter data located in featuredata.xls file.

Area Fraction

Area

Perimeter

From these primary measurement parameter data, which are all totals for the selected image frame ROI, a number of secondary derived parameters can be calculated using the following calculations:

Fullness ratio=Sqrt.(area/convex area)

Convexity=convex perimeter/perimeter

Finally, the secondary parameters are combined with area fraction to calculate the PCV parameter:

PCV=(Area/Perimeter×Convexity/Fullness ratio)/Area Fraction

PCV will be in units of a linear distance, so the type of units chosen (e.g. millimeters vs. inches) for image pixel calibration will affect the magnitude of the resulting PCV.

Multiple replicates from a single sample can be performed during a single execution of the QUIPS algorithm. Primary dimensional data will be transferred to the EXCEL spreadsheets for each replicate. Between each replicate, a new sample is placed onto the auto-stage and adjusted via a joystick for image acquisition and analysis. The final sample mean PCV parameter is based on an N=5 analysis from five, separate, product specimen subsamples. A comparison between different samples can be performed using a Student's T analysis at the 90% confidence level.

The Pattern Complexity Value method returns a complexity value for a given graphic wherein the more complex the graphic the lower the complexity value. Likewise, the less complex the graphic the higher the complexity value. In other words, graphics having a lower complexity value are more complex than graphics having a relatively higher complexity value (i.e., the PCV is inversely proportional to the complexity of the graphic). The Pattern Complexity Value is measured along a transverse axis on the absorbent article.

### Color Difference

To determine differences in color and intensity between graphics, color images of the given graphic patterns can be acquired using the QUIPS algorithm described herein. These images can then be measured for their pattern component L*a*b* color values using a number of software packages. For example, image processing and analysis packages such as Matlab (v.6.5.1, release 13; Mathworks), Adobe Photoshop, and Media Cybernetics Image Pro Plus are all suitable for measuring L*a*b* values of various colored graphics within a given pattern. Color values of the various graphic can also be measured using a BYK-Gardner Color-Guide Sphere set to the d65/10 for a fluorescent illuminant reading L*a*b* color values. After calibrating the instrument, samples possessing colored graphics were placed on top of the white backing tile, which is placed on a horizontal surface. The white backing tile comes standard with the equipment: (Color reflectance) 90 by 90 mm, Avian Technologies part number ATROG-Cstm-95. The instrument is placed on the sample and held level to the tile. Four replicate readings each of L*, a*, and b* were acquired per sample and averaged.

The differences between graphic colors and brightness can be quantified by a ΔE calculation which includes each of the L*a*b* values as illustrated below:

ΔE=[(L1−L2)²+(a1−a2)²+(b1−b2)²]

Two colors are considered different if they are just-noticeably different by a consumer. This difference is sometimes estimated by just-noticeable difference or differential threshold that has been quantified by ΔE*ab greater than 2.3 as described in ASTM D2244-09b Standard Practice for Calculation of Color Tolerances and Color Differences from Instrumentally Measured Color Coordinates and the references cited therein. Although this just-noticeable difference is sufficient, a larger difference is preferable because the additional contrast between colors is believed to enhance the perceived complexity of the color graphics.

### EXAMPLES

All colors described within the Examples are colors identified by the Color Matching System commercially available from Pantone, Inc. (Carlstadt, N.J.).

### Example 1

FIG. 2 illustrates one embodiment of the attachment member 68, 70 described herein. As illustrated, the substrate of the attachment member includes a first color, PMS7506. The first side section of the attachment member is printed with the illustrated pattern utilizing the color PMS4685. The second side section of the attachment member is printed with the illustrated pattern utilizing color PMS263. The attachment member was attached to an absorbent article as described above and tested to determine a Pattern Complexity Value and a Color Difference Value. The first side section had Pattern Complexity Value of 1.22 mm and the second side section had a Pattern Complexity Value of 0.12 mm The ratio of the second Pattern Complexity Value to the first Pattern Complexity Value is 1:10.2. The color Difference Value between the first side and the second side is 10.87.

### Example 2

FIG. 3 illustrates another embodiment of the attachment member 68, 70 described herein. As illustrated, the substrate of the attachment member includes a first color, PMS7506. All colors described within the Examples are colors identified by the Color Matching System commercially available from Pantone, Inc. (Carlstadt, N.J.). The first side section of the attachment member is printed with the illustrated pattern utilizing the color PMS4685. The second side section of the attachment member is printed with the illustrated pattern utilizing color PMS263. The attachment member was attached to an absorbent article as described above and tested to determine a Pattern Complexity Value and a Color Difference Value. The first side section had Pattern Complexity Value of 0.37 mm and the second side section had a Pattern Complexity Value of 0.12 mm The ratio of the second Pattern Complexity Value to the first Pattern Complexity Value is 1:3.08. The color Difference Value between the first side and the second side is 17.22.

### Example 3

FIG. 4 illustrates another embodiment of the attachment member 68, 70 described herein. As illustrated, the substrate of the attachment member includes a first color, PMS7506. All colors described within the Examples are colors identified by the Color Matching System commercially available from Pantone, Inc. (Carlstadt, N.J.). The first side section of the attachment member is printed with the illustrated pattern utilizing the color PMS4685. The second side section of the attachment member is printed with the illustrated pattern utilizing color PMS263. The attachment member was attached to an absorbent article as described above and tested to determine a Pattern Complexity Value and a Color Difference Value. The first side section had Pattern Complexity Value of 0.76 mm and the second side section had a Pattern Complexity Value of 0.10 mm The ratio of the second Pattern Complexity Value to the first Pattern Complexity Value is 1:7.6. The color Difference Value between the first side and the second side is 14.85.

### Example 4

FIG. 5 illustrates another embodiment of the attachment member 68, 70 described herein. As illustrated, the substrate of the attachment member includes a first color, PMS cool gray 5. All colors described within the Examples are colors identified by the Color Matching System commercially available from Pantone, Inc. (Carlstadt, N.J.). The first side section of the attachment member is printed with the illustrated pattern utilizing the color PMS539. The second side section of the attachment member is printed with the illustrated pattern also utilizing color PMS539. The attachment member was attached to an absorbent article as described above and tested to determine a Pattern Complexity Value and a Color Difference Value. The first side section had Pattern Complexity Value of 1.08 mm and the second side section had a Pattern Complexity Value of 5.22 mm. The ratio of the second Pattern Complexity Value to the first Pattern Complexity Value is 4.8:1. The color Difference Value between the first side and the second side is 9.2.

It should be understood that various other embodiments, modifications, and equivalents to the embodiments of the absorbent article described herein which, after reading the description herein, may suggest themselves to those skilled in the art without departing from the scope and spirit of the present claims.

What is claimed is:

1. An attachment member for a disposable absorbent article comprising:
a. a first side, the first side section securely attached to the disposable absorbent article, the first side having a first graphic;
b. a second side, the second side releasably attached to the disposable absorbent article, the second side having a second graphic; and
c. the first graphic has a first Pattern Complexity Value and the second graphic having a second Pattern Complexity Value, wherein the ratio of the second Pattern Complexity Value to the first Pattern Complexity Value is between 1:50 and 50:1.

2. The attachment member of claim 1 wherein the ratio of the second Pattern Complexity Value to the first Pattern Complexity Value is between 1:10 and 10:1.

3. The attachment member of claim 1 wherein the first graphic has a first color and the second graphic has a second color, and the first color and the second color are different.

4. The attachment member of claim 3 wherein a Color Difference Value between the first side and the second side is at least 2.0.

5. The attachment member of claim 4 wherein a Color Difference Value between the first side and the second side is between 3.0 and 20.

6. The attachment member of claim 4 wherein the first color on the first graphic corresponds with a color on a front panel of the disposable absorbent article.

7. The attachment member of claim 1 wherein the terminal edge of the second side of the attachment member has a shaped edge.

8. An attachment member for a disposable absorbent article comprising:
a. a first side, the first side section securely attached to the disposable absorbent article, the first side having a first graphic;
b. a second side, the second side releasably attached to the disposable absorbent article, the second side having a second graphic; and
c. the first graphic has a first color and the second graphic has a second color, and the first color and the second color are different.

9. The attachment member of claim 8 wherein a Color Difference Value between the first side and the second side is at least 2.0.

10. The attachment member of claim 9 wherein a Color Difference Value between the first side and the second side is between 3.0 and 20.

11. The attachment member of claim 8 wherein the first color on the first graphic corresponds with a color on a front panel of the disposable absorbent article.

12. The attachment member of claim 8 wherein the terminal edge of the second side of the attachment member has a shaped edge.

13. The attachment member of claim 8 wherein the first graphic has a first Pattern Complexity Value and the second graphic having a second Pattern Complexity Value, wherein the ratio of the second Pattern Complexity Value to the first Pattern Complexity Value is between 1:50 and 50:1.

14. The attachment member of claim 13 wherein the ratio of the second Pattern Complexity Value to the first Pattern Complexity Value between 1:10 and 10:1.

15. An absorbent article comprising:
a. a front waist region;
b. a back waist region;
c. an absorbent assembly secured to said front and back waist regions.
d. a first attachment member having a first side and a second side, the first side section securely attached to the disposable absorbent article, the first side having a first graphic; the second side releasably attached to the disposable absorbent article, the second side having a second graphic; and
e. the first graphic has a first Pattern Complexity Value and the second graphic having a second Pattern Complexity Value, wherein the ratio of the second Pattern Complexity Value to the first Pattern Complexity Value is between 1:50 and 50:1.

16. The absorbent article of claim 15 wherein the ratio of the second Pattern Complexity Value to the first Pattern Complexity Value is between 1:10 and 10:1.

17. The absorbent article of claim 15 wherein the first graphic has a first color and the second graphic has a second color, and the first color and the second color are different.

18. The absorbent article of claim 17 wherein a Color Difference Value between the first side and the second side is at least 2.0.

19. The absorbent article of claim 18 wherein a Color Difference Value between the first side and the second side is between 3.0 and 20.

20. The absorbent article of claim 17 wherein the first color on the first graphic corresponds with a color on a front panel of the disposable absorbent article.

21. The absorbent article of claim 15 wherein the terminal edge of the second side of the attachment member has a shaped edge.
